(19) United States
(12) Patent Application Publication
(10) Pub. No.: US 2025/0003994 A1
(43) Pub. Date: Jan. 2, 2025

(54) DEVICE AND METHOD FOR TREATING TISSUE OXYGEN DEFICIENT CONDITIONS

(71) Applicant: IP SCIENCE LIMITED, Cambridge (GB)

(72) Inventors: Ivan PETYAEV, Cambridge (GB); Robert HARPER, Mount Laurel, NJ (US); Thomas CHRISTIANI, Cherry Hill, NJ (US); George TEMENG, Cherry Hill, NJ (US)

(21) Appl. No.: 18/833,147

(22) PCT Filed: Nov. 26, 2022
(86) PCT No.: PCT/IL2022/051257

§ 371 Date: Jul. 25, 2024

Related U.S. Application Data
(60) Provisional application No. 63/283,319, filed on Nov. 26, 2021.
(60) Provisional application No. 63/283,329, filed on Nov. 26, 2021.

(51) Int. Cl. G01N 33/92; A61K 31/01; A61K 31/047; A61K 31/122; B01L 3/00

(52) U.S. Cl. CPC G01N 33/92; A61K 31/01; A61K 31/047; A61K 31/122; B01L 3/5023; B01L 2200/16; B01L 2300/0681; B01L 2300/069; G01N 2800/7038

(57) ABSTRACT

The present disclosure relates generally to lipid oxygen, and blood lipoprotein oxygen in particular, as a new treatment target and as screen for developing new therapeutic treatments, which in turn can support and improve lipid oxygen level. The present disclosure further generally relates to dry chemistry-based assays for catalymetric measurement of oxygen in biological samples, in particular to point-of-care tests for catalymetric measurement of oxygen in biological samples.

### FIELD OF INVENTION

The present disclosure relates generally to lipid oxygen, and blood lipoprotein oxygen in particular, as a new treatment target and as screen for developing new therapeutic treatments, which in turn can support and improve lipid oxygen level. The present disclosure further generally relates to dry chemistry-based assays for catalymetric measurement of oxygen in biological samples, in particular to point-of-care tests for catalymetric measurement of oxygen in biological samples.

### BACKGROUND

Molecular oxygen (O₂) is essential for metabolic functions in human and animal cells. Thus, the lack of sufficient O₂ is detrimental to the well-being of a subject. For example, the deficiency of O₂ in body tissues results in reduction of their respiration, energy production and organ functions, which can lead to transient or chronic hypoxia and contribute to development of diseases.

Even at a subclinical level, (in persons not displaying clinical symptoms) insufficient tissue oxygenation, and reduction in respiration may lead to the gradual development of negative pathological conditions.

Presently, there are a number of physical treatments and clinical approaches aiming to support and improve the oxygen content in the body. These include a) increasing the level of O₂ in inhaled gas; b) improving/opening air oxygen pathways (in the case of pathological respiratory conditions) or blood flow (in the case of cardiovascular conditions) by surgical procedures; and c) hyperbaric oxygenation, which directly targets the level of O₂ in tissues.

In addition, two existing therapeutic approaches-employing a chemical treatment are also available. These include a) treatments that target the level of haemoglobin in the blood, or b) treatments aiming at increasing the passive flow/diffusion of O₂ dissolved in the aqueous electrolyte phase of blood or interstitial fluid.

Haemoglobin (Hb) in erythrocytes is the main transporter of oxygen in the blood. However, erythrocytes cannot pass the capillary wall to directly deliver O₂ to tissue cells.

It has been demonstrated that plasma lipoproteins can carry significant amounts of oxygen gas. This property is enabled by the hydrophobic crystalline structure of lipids, which provides a favorable environment for O₂ solubility to a greater degree than that found in an aqueous medium. Apart of oxygen-transporting lipoproteins, other lipid structures (lipid droplets, micelles, cellular membranes etc.) are essential for tissue O₂ storage, cellular respiration and energy production.

However, this significant pool of oxygen has not been sufficiently targeted prior to the herein disclosure—the oxygen content of lipids in biological systems.

To date there are two main methods for assessing the level of molecular oxygen, O₂, in biological tissues.

The first is polarography, which utilizes a Clark electrode/probe and an electronic base reader, quantifying the electrode/probe signal. In clinical practice, it used in various gas analyzers and exclusively for whole blood analysis. Polarography can be used to measure O₂ in other body tissues, however it is an invasive procedure, hence it is only used for research purposes. Moreover, polarography measures the total pool of O₂ in the blood, or in the analyzed biopsy sample, without differentiating between intracellular and extracellular oxygen. Although these pools are interchangeable, their separate assessment may be of particular interest, especially in order to detect the underlying cause of disrupted O₂ tissue supply and tissue oxygenation/respiration, which may be paramount for deciding on an optimal treatment for correcting and/or for evaluating preventive measures that may be taken in order to prevent or inhibit tissue hypoxia.

The second method is catalymetry, which measures a rate of 02-depended chemical reactions as a function of its concentration using an electronic reader quantifying the concentration of the end product. This method is beneficial in that it measures the extracellular pool of oxygen, e.g., in blood plasma, serum, interstitial fluid or other acellular biological fluids.

However, both methods have significant drawbacks.

Polarography O₂ gas analysis requires complex hospital equipment, including the unique oxygen electrode and the electronic reader. In addition, to use this equipment and analyze the results a trained medical person is needed.

Catalymetry of O₂ is significantly more affordable, however it still requires clinical laboratory equipment and trained technicians to perform the analysis. Moreover, since it is based on “wet chemistry” it requires use of freshly prepared solutions and reagents, which again necessitates laboratory ware, pipetting and other consumables. In addition, treating and separating blood for the test, preparation of fresh reagents, and the analysis of the readouts, is time-consuming, typically around an hour.

Therefore, a twofold need exists for a method that (i) utilizes the O₂ level in body lipids, either present in fluids, in the form of lipoproteins, or in tissues, as a clinical diagnostic tool, which can be used to assess the health risk or severity of conditions caused by O₂ deficiency, and (ii) enables the development of treatments aimed at promoting healthy tissue O₂ levels in a subjects' body. Additionally, there remains a need, for an affordable, fast point of care assay for assessing the level of molecular oxygen in biological tissues.

### SUMMARY OF THE INVENTION

According to some embodiments, there is provided herein a method for measuring and/or monitoring tissue oxygen (O₂) supply capability of a subject by assessing lipid oxygen concentration or oxygen carrying capacity of plasma lipoproteins (OCCL) in a biological sample taken from clinically healthy individuals and/or from those who have potential health risk factors.

Advantageously, the herein disclosed method enables straightforward assessment and monitoring of tissue oxygenation in healthy persons and/or in those who have potential health risk factors, to detect its impairment in asymptomatic individuals to warrant corrective measures to prevent development of conditions resulting from tissue O₂ deficiency.

According to some embodiments, there is provided herein a method for measuring and/or monitoring tissue oxygen supply capability of a subject by assessing OCCL in a biological sample taken from patients with acute tissue ischemic conditions, myocardial infarction (MI), and compared with age and gender matched clinically healthy subjects. In parallel, the concentration of total oxygen in blood PaO₂ could be measured in both groups of participants.

Advantageously, the herein disclosed method enables straightforward monitoring/assessing conditions resulting from tissue O₂ deficiency.

Advantageously, the method herein disclosed shows that measurement of the OCCL is a much more informative to assess oxygen tissue delivery than PaO₂, because the level of OCCL was reduced by about 50% in patients with severe myocardial hypoxia as compared to healthy individuals, while the level of PaO₂ was unchanged in both populations.

According to some embodiments, there is provided herein a method for using measurement of the OCCL in biological samples taken during different phases of tissue hypoxic impact, from its acute phase and through recovery phases. According to some embodiments the method may allow assessment of a success or lack of success in recovery of reduced tissue oxygenation level.

Advantageously, the herein disclosed method enables the development of therapeutic methods for increasing the O₂ tissue delivery level in an affected subject, allowing for preventing, ameliorating and/or treating a variety of ailments.

As a further advantage, the therapeutic methods available conveniently include dietary and/or other life-style changes, and/or administration of readily available nutraceuticals, and/or repurpose existing pharmaceuticals and/or development of new pharmaceuticals which may be administered directly to support and/or restore healthy tissue O₂ supply level in a subject.

Some embodiments relate to a device for measuring a concentration of molecular oxygen in a biological sample, the device including a housing comprising a plurality of membranes, wherein the plurality of membranes includes a separation membrane configured to separate components in the biological sample, and a reagent membrane configured to facilitate an oxygen dependent reaction, wherein the oxygen dependent reaction is indicative of the concentration of molecular oxygen in the biological samples.

According to some embodiments, the biological sample may be selected from the group consisting of a whole blood sample, plasma, serum, cerebrospinal fluid, interstitial fluid, milk, a cerumen sample, a skin sebum, or its other exfoliated material, a skin or other tissue swab, or any combination thereof.

According to some embodiments, the biological sample is a whole blood sample, plasma or serum. According to some embodiments, the biological sample includes lipids and/or lipoproteins.

According to some embodiments, the separation membrane includes a first membrane and a second separation membrane.

According to some embodiments, the first separation membrane includes a D23 membrane. According to some embodiments, the second separation membrane includes a mixed matrix membrane (MMM).

According to some embodiments, the plurality of membranes further includes a blood spreading membrane.

According to some embodiments, the reagent membrane includes an oxidating agent and a reducing agent. According to some embodiments, the oxidizing agent includes a quinone derivative, e.g., menadione.

According to some embodiments, the device may be configured such that the biological samples flow vertically from the blood spreading membrane, through the first membrane and second separation membrane to the reagent membrane.

According to some embodiments, the device may be configured to indicate indicative of an oxygen carrying capacity, oxygen carrying capacity reserve, or oxygen take up ability in the biological sample. According to some embodiments, the device may be configured for use in assessment of predisposition and/or resistance to hypoxic conditions or diseases. According to some embodiments, the device may be configured for use in diagnosis of hypoxia-associated asymptomatic or symptomatic pathologies.

According to some embodiments, the device may be configured for use in assessment and/or monitoring of effects of an administered/consumed dietary/food supplements, and or nutraceuticals, or pharmaceuticals, or medical procedures, or dietary and/or life-style factor, or a combination thereof. According to some embodiments, the device may be configured for use in development of a functional food or beverage, or nutraceuticals, or pharmaceuticals products configured to support tissue oxygenation, to prevent and/or to treat tissue hypoxic conditions. According to some embodiments, the device may be configured for use in assessment of integrity and/or quality of a lipid containing food, beverage, nutraceutical, or pharmaceutical, or industrial products.

According to some embodiments, the device may further include a reagent configured to change its color in response to an oxygen dependent reaction.

According to some embodiments, the device may be configured to be functionally associated with a processor and/or App configured to determine the intensity of the color based on image recognition and/or analysis. According to some embodiments, the processor and/or App is further configured to present a user with an indication of the concentration of oxygen in the biological sample based on the determined intensity.

Some embodiments relate to an assay for measuring a concentration of molecular oxygen in a biological sample, the assay including:

loading a biological sample on a cassette, the cassette comprising a plurality of layers/membranes, wherein the plurality of membranes including:

a separation membrane configured to separate components in the biological sample, and

a reagent membrane/layer configured to facilitate an oxygen dependent reaction, the oxygen dependent reaction indicative of the concentration of molecular oxygen in the biological samples,

allowing vertical flow through of the biological sample through the plurality of layers/membranes until reaching the reagent membrane.

According to some embodiments, there is provided herein a method for measuring and/or monitoring a lipid and/or lipoprotein oxygen concentration and/or O₂ level supplied to a tissue and its uptake capability in a subject. According to some embodiment, the method may comprise measuring the OCCL in a biological sample of the subject, using a biochemical, electro-chemical, chemical and/or physical method or assay.

According to some embodiments, assessing the O₂ level may comprise subjecting the biological sample to a reagent configured to facilitate an oxygen dependent reaction, the oxygen dependent reaction indicative of the concentration of molecular oxygen in the tested biological samples.

According to some embodiments, there is provided a device and wet and/or dry chemistry assay for measuring a concentration of molecular O₂ in lipids and/or lipoprotein particles in biological samples including/utilizing components, such as one or more membranes, optionally impregnated with reagents, the interactions of which may be sensitive to and/or may depend on oxygen.

Advantageously, the device is low-cost and/or disposable, which may not require use of hospital and/or clinical laboratory equipment. Moreover, the device may not require any technical expertise and the O₂ measurement may be conducted in its entirety by any user (e.g., patient, family member, or caregiver), thus facilitating point-of-care use.

As a further advantage, the test performed using the device/assay may be fast, typically less than 15 minutes, less than 10 minutes, or less than 5 minutes. Each possibility is a separate embodiment.

According to some embodiments, the biological sample may be a blood plasma, serum, cerebrospinal fluid, interstitial fluid, synovial fluid, milk, sebum, exfoliated material, biopsy, or other biological sample. Each possibility is a separate embodiment.

According to some embodiments, changing the oxygen-related condition may comprise increasing a tissue oxygen demanding physical work, mental work, physical stress, and/or mental stress on the subject. According to some embodiments, increasing the stress on the subject may include physical work and/or exercise. According to some embodiments, the physical exercise may include running on a treadmill. According to some embodiments, increasing the stress on the subject may include inducing a transient stagnant ischemia test.

According to some embodiments, the subject may be at risk of a tissue oxygenation deficiency or of a chronic or acute hypoxic event. According to some embodiments, the subject may be pregnant.

According to some embodiments, the subject may suffer from coronary artery disease, unstable angina, acute myocardial infarction, peripheral tissue or organ ischaemia, metabolic syndrome, obesity, a cardiovascular disease, a cerebrovascular disease, a vascular occlusive disease, diabetes, cancer, dementia, neurodegenerative diseases, a bacterial, viral or fungal infection, a respiratory disease, an autoimmune disease, or any combination thereof. Each possibility is a separate embodiment.

According to some embodiments, the subject may suffer from coronary artery disease, unstable angina, acute myocardial infarction, peripheral tissue, or organ ischemia. Each possibility is a separate embodiment.

According to some embodiments, there is provided herein a method for preventing, ameliorating and/or treating a condition characterized by insufficient tissue oxygenation. According to some embodiments, the method may include administering to a subject in need thereof a therapeutic agent capable of increasing OCCL in a subject.

According to some embodiments, the treating may be conducted if the subject shows less than about a 10% increase in the OCCL when subjected to a change in an oxygen-related condition.

According to some embodiments, the measuring the OCCL may include performing a biochemical, electro-chemical, chemical, and/or physical method or assay on a biological sample obtained before and/or after subjecting a subject to a change in the oxygen tissue supply and/or tissue oxygenation conditions.

According to some embodiments, the measuring may include subjecting biological samples to a reagent configured to facilitate an oxygen dependent reaction, wherein the oxygen dependent reaction may be indicative of the concentration of molecular oxygen therein.

According to some embodiments, the biological sample may be a blood plasma, serum, cerebrospinal fluid, interstitial fluid, milk, sebum, exfoliated material, biopsy or other biological sample. Each possibility is a separate embodiment.

According to some embodiments, the subject may be at risk of a tissue oxygenation deficiency, chronic or acute hypoxic event. According to some embodiments, the subject may be pregnant. According to some embodiments, the condition may be ageing.

According to some embodiments, the subject may suffer from coronary artery disease, unstable angina, acute myocardial infarction, peripheral tissue or organ ischemia, metabolic syndrome, obesity, a cardiovascular disease, a cerebrovascular disease, a vascular occlusive disease, diabetes, cancer, dementia, neurodegenerative diseases, a bacterial, viral or fungal infection, a respiratory disease, an autoimmune disease, or any combination thereof. Each possibility is a separate embodiment.

According to some embodiments, the subject may suffer from coronary artery disease, unstable angina, acute myocardial infarction, peripheral tissue, or organ ischemia. Each possibility is a separate embodiment.

According to some embodiments, the condition may be symptomatic or asymptomatic.

According to some embodiments, the therapeutic agent may be administered orally.

According to some embodiments, the therapeutic agent may be administered daily for at least 3 weeks. According to some embodiments, the therapeutic agent may be administered daily for at least 4 weeks.

According to some embodiments, the therapeutic agent may be Lycopene, Lutein, Zeaxanthin, Astaxanthin, Coenzyme Q10, or any combination thereof. Each possibility is a separate embodiment. According to some embodiments, the Lycopene may be GA Lycopene.

According to some embodiments, increasing the OCCL may comprise reducing increased systolic blood pressure, reducing increased Ankle-Brachial Index (ABI), and/or increasing reduced Endothelium-dependent flow-mediated (FMD) vasodilation, and/or improving other cardiovascular or cerebro-vascular parameters. Each possibility is a separate embodiment.

According to some embodiments, there is provided a method for identifying a therapeutical agent as being capable of increasing lipid/lipoprotein oxygen level, capacity to carry oxygen, and/or deliver oxygen to tissue cells to boost/increase tissue oxygenation level. According to some embodiments, the method may include: measuring the OCCL level in a subject before and/or at a predetermined time after administering the therapeutical agent, wherein the measuring may include assessing a level of OCCL in a blood sample and/or other biological sample using a biochemical, electro-chemical, chemical or physical method or assay, and identifying the therapeutical agent as being capable of increasing tissue oxygenation level, when an increase in OCCL is identified as a positive effect/result of the administration thereof.

According to some embodiments, the method may further include subjecting a subject to tissue oxygen demanding physical work, mental work, physical stress and/or mental stress. According to some embodiments, subjecting the subject to stress may include tissue oxygen demanding physical work, mental work, physical and/or mental exercise. Each possibility is a separate embodiment. According to some embodiments, the physical exercise may include running on a treadmill. According to some embodiments, subjecting the subject to stress may include inducing a transient stagnant ischemia test in the subject.

According to some embodiments, the therapeutical agent may include life-style changes, a nutraceutical, a functional food or beverage, a dietary product, a pharmaceutical agent, a physical treatment or procedure, or any combination thereof. Each possibility is a separate embodiment.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some, or none of the enumerated advantages.

### BRIEF DESCRIPTION OF THE FIGURES

The invention will now be described in relation to certain examples and embodiments with reference to the following illustrative figures.

FIG. 1 schematically illustrates a point-of-care device for measuring the concentration of oxygen in a biological sample, according to some embodiments.

FIG. 2 shows reagent intensity obtained when testing oxygen associated lipid/lipoproteins in blood plasma at different dilutions, as compared to two negative controls (no sample and water) and to a positive control (1 M KO₂). The cassette included the following membranes primary=D23, Secondary=MMM, Reagent=Cellulose Filter Paper. The analyzed sample volume was 30 μl, observation time=5 minutes.

FIG. 3 shows quantified luminance of the test results of FIG. 2 obtained by measuring the intensity of light emitted from a surface per unit area in a given direction.

FIG. 4 is an exemplary flowchart of the herein disclosed method measuring and/or monitoring a subject's oxygen lipid/lipoprotein uptake capability, according to some embodiments.

FIG. 5 is an exemplary flowchart of the herein disclosed method for preventing, ameliorating and/or treating tissue hypoxia, according to some embodiments.

FIG. 6 is an exemplary flowchart of the herein disclosed method for identifying a therapeutical agent as being capable of increasing lipid/lipoprotein oxygen uptake capability, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances 10%, or in some instances ±5%, or in some instances ±1%, or in some instances ±0.1% from the specified value, as such variations are appropriate to perform the disclosed methods. Each possibility is a separate embodiment.

Although it has been demonstrated that plasma lipoproteins can carry significant amounts of oxygen gas, this significant pool of oxygen has not been sufficiently targeted prior to the herein disclosure, which advantageously provides method for monitoring the oxygen carrying capacity of lipid/lipoprotein of biological samples, methods for treating tissue hypoxia and/or improving the oxygen carrying capacity of lipid/lipoprotein, as well as assays/screens for use in development and or identification of therapeutic agents capable of improving the oxygen carrying capacity of lipid/lipoprotein.

### Part 1. Method to Assess Lipid Oxygen Concentration/Tissue Oxygenation Level

According to some embodiments, there is provided herein an assay for monitoring oxygen quantitatively, using aqueous mixtures containing lipid micelles, lipid emulsions and/or artificial lipoproteins. According to some embodiments, the assay may enable monitoring the oxygen content of lipids/lipoproteins in whole blood plasma, serum, and/or other biological fluids/materials by monitoring an oxygen dependent reaction with oxygen in lipid components. According to some embodiments, the assay may measure nitro-blue-tetrazolium-detectable superoxide generated by reduced nicotinamide adenine dinucleotide (NADH) and phenazine methosulphate in the presence of diethylenetriamine penta-acetic acid.

Advantageously, the herein disclosed device and associated assay is based on “dry chemistry”, which obviates the need for specialized laboratory techniques and equipment.

Needless to say, no simple adaptation of the existing “wet” chemistry catalymetry assay for measuring extracellular O₂ in blood, into a “dry” chemistry exists. In fact, developing a point-of-care assay has hitherto been hampered by a number of technical obstacles. For example, some reagents used in the “wet” test were found not to be as stable as or as interacting with the “dry” matrixes, which may create difficulties such as undesired backgrounds and/or artifacts which may negatively influencing the oxygen catalymetry and its results.

Advantageously, these obstacles have been overcome by the hereindisclosed device and assay, which enables an indirect and non-invasive assessment of the level of oxygen in a biological sample, by measuring the level of lipid-associated oxygen in the sample, the assay being based on dry chemistry and vertical flow.

According to some embodiments, the device may include a cassette/housing configured to receive a biological sample and/or for separating its components (e.g., separating red blood cells from plasma).

According to some embodiments, the cassette may include a well configured to receive a biological sample. According to some embodiments, the well may include a plurality of membranes, such as two, three, four or more membranes. Each possibility is a separate embodiment.

According to some embodiments, the well may include a hydrophilic mesh (also referred to herein as “blood spreading membrane”), such as but not limited to a glass fiber membrane impregnated with pro-agglutinating agents. According to some embodiments, the hydrophilic mesh may be configured to cause the plasma to spread evenly across the surface of a blood separation membrane (also referred to herein as “first blood separation layer”). According to some embodiments, the blood separation membrane may serve to remove cells from the plasma sample.

Alternatively, and/or additionally, the device may further include a cell capturing membrane (also referred to herein as “second blood separation layer”), such as but not limited to a polyether sulfone membrane, which may be configured to capture any remaining agglomerated cells.

According to some embodiments, the device may further include an anisotropic reagent membrane (also referred to herein as “oxidative stress detection layer”). According to some embodiments, the anisotropic reagent membrane may be configured to generate a color-reaction indicative of the levels of oxygen in the sample. According to some embodiments, the color intensity of the anisotropic reagent membrane may be directly or inversely proportional to the oxygen concentration in the sample.

According to some embodiments, the primary separation membrane may be a whole blood separation membrane, which may also be known as D23. According to some embodiments, the primary separation membrane may be prepared by dissolving a synthetic water-soluble polymer (e.g., polyvinyl alcohol, etc.) in water (e.g., by heating). After dissolving, one or more or all of a surfactant(s), buffer(s), sugar(s), stabilizer(s) salt(s) and sugar alcohol(s) may be added. The solution may then be pH adjusted and isopropanol (IPA) may be added.

According to some embodiments, the water-soluble synthetic polymer may be or may include PVA

According to some embodiments the surfactant may be a non-ionic surfactant. According to some embodiments the surfactant may be or may include surfactant 10G (glycidol surfactant), glycerol monostearate, sorbitan monostearate, poloxamer, polysorbate, cetyl alcohol, etc. Each possibility is a separate embodiment.

According to some embodiments the buffer may be a zwitterionic buffer e.g., piperazine, etc. According to some embodiments, the buffer may be any buffer capable of forming radicals. According to some embodiments, the buffer may be 1,4-piperazinediethanesulfonic acid sodium salt (PIPES sodium salt).

According to some embodiments, the sugar may be any sugar having a high water retention capability. According to some embodiments, the sugar may be trehalose, sucralose, sucrose, etc. Each possibility is a separate embodiment.

According to some embodiments, the stabilizer may be a protein stabilizer. According to some embodiments, the stabilizer may be neo protein saver (NPS).

According to some embodiments, the salt may be NaCl, NaI, NaBr, KCl, KI, KBr, etc. Each possibility is a separate embodiment.

According to some embodiments, the sugar alcohol may be any sugar alcohol capable of elevating blood plasma osmolality and/or of enhancing flow of water from tissues. According to some embodiments, the sugar alcohol may be mannitol, sorbitol, erythritol, etc. Each possibility is a separate embodiment.

According to some embodiments, the secondary separation membrane may be a mixed matrix membrane (MMM).

According to some embodiments, the secondary separation membrane includes a water-soluble synthetic polymer, thickener(s) and/or emulsifier(s) and/or softener(s), and/or chelating agent(s), and/or surfactant(s). Each possibility is a separate embodiment.

According to some embodiments, the water-soluble polymer may be synthetic. According to some embodiments, the water-soluble synthetic polymer may be or may include polyvinyl alcohol (PVA).

According to some embodiments the thickener(s) and/or emulsifier(s) and/or softener(s) may be or may include monosodium phosphate (phosphate monobasic) and/or disodium phosphate (phosphate dibasic) and/or carboxymethyl cellulose (CMC). Each possibility is a separate embodiment.

According to some embodiments the chelating agent may be any agent configured to prevent blood samples from clotting. According to some embodiments the chelating agent may be or may include ethylenediamine tetraacetic acid (EDTA).

According to some embodiments the surfactant may be a non-ionic surfactant. According to some embodiments the surfactant may be or include surfactant 10G (glycidol surfactant), glycerol monostearate, sorbitan monostearate, poloxamer, polysorbate, cetyl alcohol, etc. Each possibility is a separate embodiment.

According to some embodiments, the reagent membrane may be a cotton linter membrane. According to some embodiments, the reagent membrane may include more than one (such as 2, 3, 4, etc.) cotton linter membrane coatings.

According to some embodiments, the first cotton linter membrane coating may include excipients, polymer(s), alcohol(s) and surfactant(s). Each possibility is a separate embodiment.

According to some embodiments, the excipient may be a non-ionic polymer. According to some embodiments, the non-ionic polymer may include hydroxypropylcellulose. According to some embodiments, the non-ionic polymer may include or be Klucel™ EF.

According to some embodiments, the alcohol may be or may include methanol, ethanol, n-propanol, i-propanol, n-butanol, t-butanol, etc. Each possibility is a separate embodiment.

According to some embodiments the surfactant may be a non-ionic surfactant. According to some embodiments the surfactant may be or include 2-[4-(2,4,4-trimethylpentan-2-yl)phenoxy]ethanol (e.g., Triton X-100), glycidol surfactant, glycerol monostearate, sorbitan monostearate, poloxamer, polysorbate, cetyl alcohol, etc. Each possibility is a separate embodiment.

According to some embodiments, the first coating may include or may be impregnated with one or more reagents configured to generate an oxygen dependent reaction. According to some embodiments, the first coating may include or may be impregnated with an oxidizing agent. According to some embodiments, the oxidizing agent may be or may include a quinone derivative. According to some embodiments, the oxidizing agent may be or may include menadione.

According to some embodiments, the first coating may include or may be impregnated with a reducing agent. According to some embodiments, the reducing agent may be or may include NADH. According to some embodiments, the reagents may further include one or more of a lectin, sodium nitrite, glutathione or any combination thereof. Each possibility is a separate embodiment.

According to some embodiments, the second cotton linter membrane may include cellulose derivative(s), surfactant(s), thickener(s), emulsifier(s), softener(s) or a combination thereof. Each possibility is a separate embodiment.

According to some embodiments, the cellulose derivative may be carboxymethyl cellulose (CMC).

According to some embodiments the thickener(s), emulsifier(s) or softener(s) may be or may include monosodium phosphate (phosphate monobasic) and/or disodium phosphate (phosphate dibasic). Each possibility is a separate embodiment.

According to some embodiments, the second coating may include or may be impregnated with a dye. According to some embodiments, the dye may include a molecule that may be reduced, e.g., when exposed to NADH and/or other reducing agent. According to some embodiments, the dye may be a Water Soluble Tetrazolium Salt, such as WST-4.

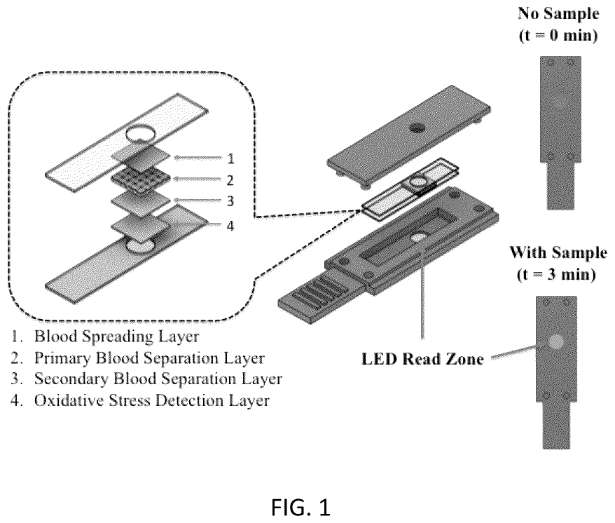

The hereindisclosed device (point-of care cassette) is schematically illustrated in FIG. 1.

According to some embodiments, the device may be functionally associated with a processor, such as a computer (e.g. of a doctor's computer), a mobile computing device (e.g., cellular phone, tablet, smart watch, smart glasses, VR device, etc.) including an App, or a dedicated device, configured to determine and/or quantify the intensity of the color reaction and/or to provide an indication of the concentration of molecular oxygen in the biological sample.

According to some embodiments, the processor may perform AI guided image analysis based upon which the color intensity may be determined and/or quantified.

According to some embodiments, the processor may further be configured to provide a recommendation, such as prompting the user to contact a doctor, or recommending the user to increase the dose of an administered drug, consumed nutraceutical/dietary supplement (e.g., lycopene, etc.), and/or adjust diet and/or life-style, or combination thereof, based on the determined extracellular oxygen levels.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1.1—Membrane Preparation

Formulations of the reagents to impregnate membrane layers of the cassette is provide in the tables below.

TABLE 1
Primary Membrane
Primary Membrane (D23)

| Component | Mass (g) |
|---|---|
| dH₂O | 25 |
| Polyvinyl alcohol (31-50K) | 0.5 |
| Heat to dissolve | |
| Add 50 ml dH2O | |
| Surfactant 10G | 0.15 |
| PIPES sodium salt | 0.302 |
| Trehalose | 0.36 |
| NaCl | 0.876 |
| NPS (neo Protein saver) fridge | 0.1 |
| Mannitol | 5 |
| pH to 6.1 | |
| IPA | 5 ml |

QS to 100 mL

TABLE 2
Secondary Membrane
Secondary Membrane (MMM)

| Component | Mass (g) |
|---|---|
| H₂O | 80 |
| 1M phosphate monobasic | 0.5 |
| 1M phosphate dibasic | 1.5 |
| Polyvinyl alcohol | 0.35 |
| EDTA | 0.074 |
| Surfactant 10G | 0.15 |

pH to 6.5
QS to 100 mL

TABLE 3
Reagent Membrane (Cotton Linter)
1^{st} Coat
First Coating of Reagent Membrane

| Component | Mass (g) |
|---|---|
| Klucel EF (hydroxypropylcellulose) | 3 |
| Methanol | 100 |
| Triton X-100 (2-[4-(2,4,4-trimethylpentan- | 0.15 |
| 2-yl)phenoxy]ethanol) | |
| QS to 100 mL | |
| Working Solution | |
| Component | Mass (g) |
| Menadione | 600 |

Working Solution (per 10 mL)

| | |
|---|---|
| Component | Mass (g) |
| Lectin | 5 |
| Sodium Nitrite | 200 |
| Glutathione | 3 |
| NADH | 7.63 |

TABLE 4

| Second Coating of Reagent Membrane | |
|---|---|
| Reagent Membrane (Cotton Linter) | |
| 2^{nd} Coat | |
| Component | Mass (g) |
| H2O | 80 |
| CMC (Carboxymethyl cellulose) | 0.35 |
| Triton X-100 (2-[4-(2,4,4-trimethylpentan- | 0.2 |
| 2-yl)phenoxy]ethanol) | |
| 1M phosphate monobasic | 2 |
| 1M phosphate dibasic | 3 |
| pH to 8.0 | |
| QS to 100 mL | |
| Working Solution | |
| Component | Concentration (μM) |
| WST-4 | 1480 |

### Example 1.2—POC for Dry Chemistry Catalymetry Assay for Detection and Quantification of Molecular Oxygen

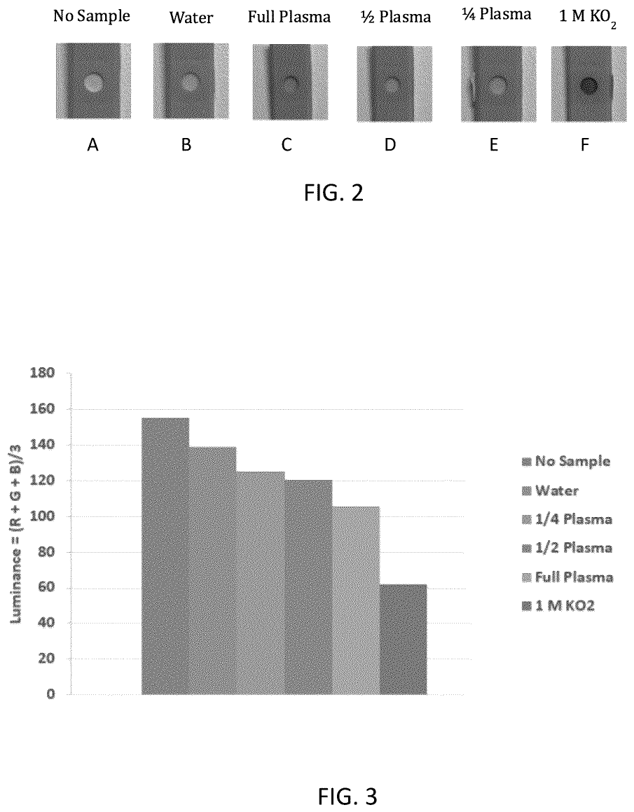

The FIG. 2 shows reagent intensity obtained when testing oxygen associated lipid/lipoproteins in blood plasma at different dilutions (full plasma (C), half plasma (D) and quarter plasma (E)), as compared to two negative controls (no sample (A) and water (B)) and to a positive control (1 M KO₂ (F)). As seen from the figure, at higher plasma lipoprotein concentrations, a more intense color was obtained. A similar observation was made when the intensity was quantified, as shown in FIG. 3.

### Part 2. Lipid Oxygen as a Treatment Target

As demonstrated, According to some embodiments, oxygen in plasma lipoproteins may be significantly reduced during physical stress and/or in acute clinical hypoxia. This indicates that lipid/lipoprotein associated oxygen may be an important source of oxygen for exercising skeletal and heart muscles, and even the whole body in clinical hypoxic conditions. According to some embodiments, lipid, and in particular lipoprotein, associated oxygen may be an important treatment target required for improving resistance to hypoxic conditions and/or for treating tissue hypoxia.

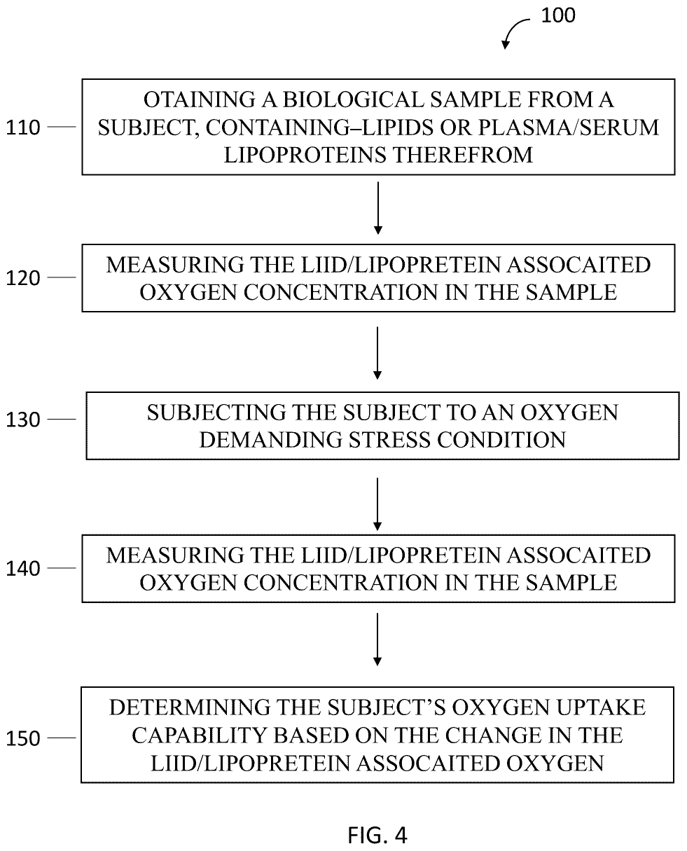

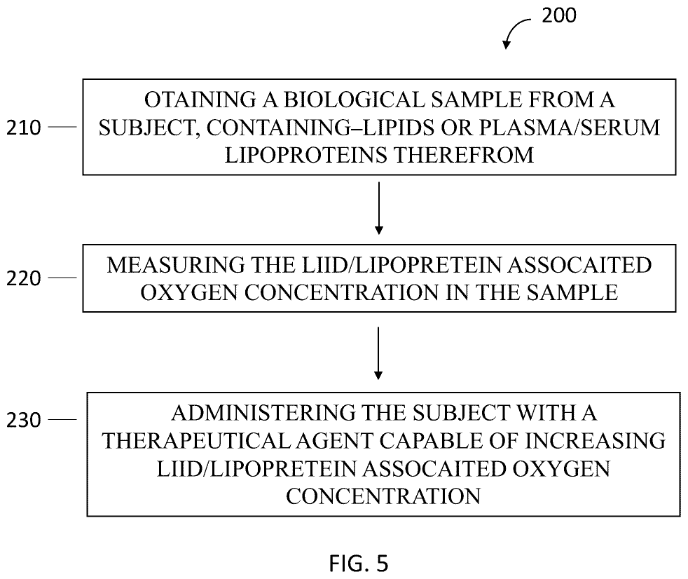

Reference is now made to FIG. 4, which schematically illustrates the flow of the herein disclosed method 100 for measuring and/or monitoring a lipid/lipoprotein oxygen concentration and/or uptake capability of a subject. It is understood that while the steps of method 100 are depicted in a particular order, some steps are sequential, but others may be performed in another order or simultaneously with another step. One of ordinary skill in the art will readily understand which steps must be performed in the indicated order and which steps may be performed at different stages or in another order of the method 100.

In step 110 a blood sample (or other biological sample) was obtained from a subject, lipid or blood plasma/serum lipoproteins extracted therefrom.

In step 120 the lipid/lipoprotein associated oxygen concentration in the sample is measured, for example by chemical testing utilizing reagents, capable of causing an oxygen-dependent reaction. As a non-limiting example, the reagent may be any one or more of reduced nicotinamide adenine dinucleotide (NADH), phenazine methyl sulfate (PMS) and nitro blue tetrazolium chloride (NBT), and the level of oxygen in the sample may be assessed, based on the oxygen dependent reduction of the reagent, which reduction causes a difference in its absorption that can be measured using spectroscopy.

In step 130, the subject is exposed to an oxygen requiring stress condition. As a non-limiting example, the subject may be requested to perform physical exercise (e.g., a treadmill test). As another non-limiting example, the subject may be subjected to a transient stagnant ischemia test (e.g., by occlusion of the brachial artery).

In step 140 the lipid/lipoprotein associated oxygen concentration in the sample is once again measured.

In step 150 the subject's oxygen uptake capability is determined based on a change (or lack thereof) in the lipid/lipoprotein associated oxygen concentration, as a result of the stress condition.

Reference is now made to FIG. 5, which schematically illustrates the flow of the herein disclosed method 200 for preventing, ameliorating and/or treating a condition characterized by insufficient tissue oxygenation. It is understood that while the steps of method 200 are depicted in a particular order, some steps are sequential, but others may be performed in another order or simultaneously with another step. One of ordinary skill in the art will readily understand which steps must be performed in the indicated order and which steps may be performed at different stages or in another order of the method 200.

In step 210 a blood sample (or other biological sample) was obtained from a subject, lipid or blood plasma/serum lipoproteins extracted therefrom.

In step 220 the lipid/lipoprotein associated oxygen concentration in the sample is measured for example by chemical testing utilizing reagents, capable of causing an oxygen-dependent reaction. As a non-limiting example, the reagent may be any one or more of reduced nicotinamide adenine dinucleotide (NADH), phenazine methyl sulfate (PMS) and nitro blue tetrazolium chloride (NBT), and the level of oxygen in the sample may be assessed, based on the oxygen dependent reduction of the reagent, which reduction causes a difference in its absorption that can be measured using spectroscopy. According to some embodiments, the measurement is performed before and after exposing the subject to a stress condition as essentially described with regards to FIG. 4.

In case it is determined that the subject has low tissue oxygenation capacity, the subject is administered with a therapeutic agent capable of increasing oxygen carrying capacity of plasma lipoproteins (OCCL) in a subject (step 230), whether or not clinical symptoms have manifested. It is understood to one of ordinary skill in the art, that the treatment may be provided to the subject without conducting steps 210 and 220, but rather in response to a diagnosis (e.g., coronary artery disease) or condition of the subject (e.g., pregnancy or ageing).

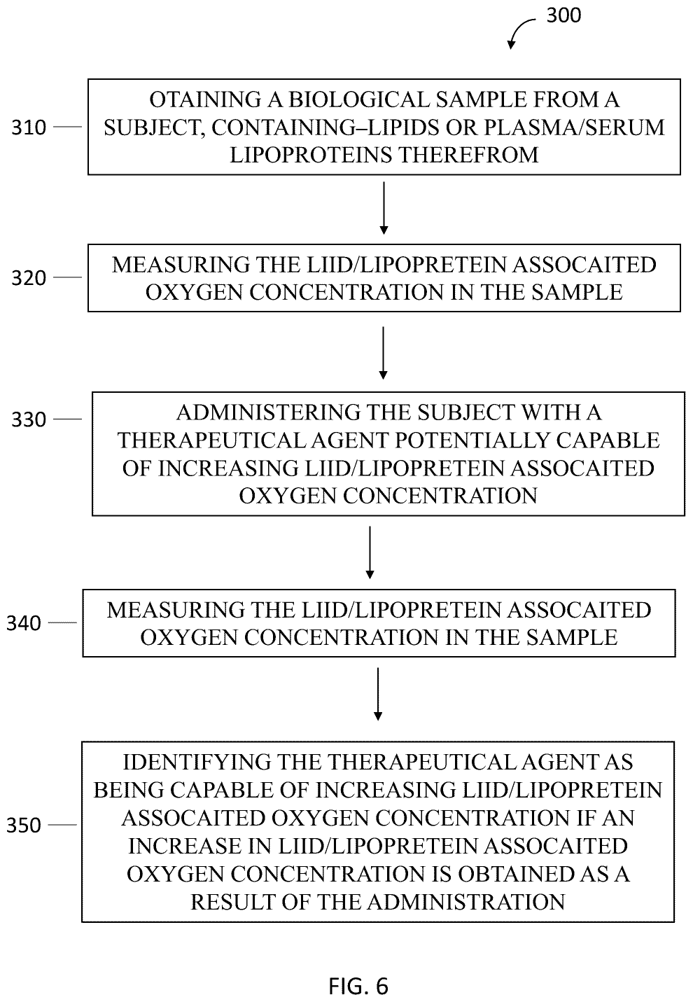

Reference is now made to FIG. 6, which schematically illustrates the flow of the herein disclosed method 300 for identifying a therapeutical agent as being capable of increasing lipid/lipoprotein oxygen uptake capability. It is understood that while the steps of method 300 are depicted in a particular order, some steps are sequential, but others may be performed in another order or simultaneously with another step. One of ordinary skill in the art will readily understand which steps must be performed in the indicated order and which steps may be performed at different stages or in another orders of the method 300.

In step 310 a blood sample (or other biological sample) is obtained from a test subject, lipid or blood plasma/serum lipoproteins extracted therefrom.

In step 320 the lipid/lipoprotein associated oxygen concentration in the sample is measured for example by chemical testing utilizing reagents, capable of causing an oxygen-dependent reaction. As a non-limiting example, the reagent may be any one or more of reduced nicotinamide adenine dinucleotide (NADH), phenazine methyl sulfate (PMS) and nitro blue tetrazolium chloride (NBT), and the level of oxygen in the sample may be assessed, based on the oxygen dependent reduction of the reagent, which reduction causes a difference in its absorption that can be measured using spectroscopy. According to some embodiments, the measurement is performed before and after exposing the subject to a stress condition as essentially described with regards to FIG. 4. In step 330 subject is administered with a therapeutic agent potentially capable of increasing oxygen carrying capacity of plasma lipoproteins (OCCL).

In step 340 the lipid/lipoprotein associated oxygen concentration in the sample is measured, as described with regards to step 320. According to some embodiments, the measurement is performed before and after exposing the subject to a stress condition as essentially described with regards to FIG. 4.

In step 350 the therapeutical agent is identified as being capable of increasing tissue oxygen uptake capability, if an increase in OCCL is identified as a result of the administration thereof.

In addition to the exemplary aspects and embodiments described above, further aspects and embodiments will become apparent by reference to the figures and by study of the following detailed descriptions.

The following examples are presented in order to illustrate some embodiments of the invention more fully. They should in no way be construed, however, as limiting the broad scope of the invention. One skilled in the art can readily devise many variations and modifications of the principles disclosed herein without departing from the scope of the invention.

### Example 2.1—Cardiovascular Stress Test

Treadmill tests are commonly used to assess cardiovascular reserves in healthily individuals or to monitor the extent of ischemia, hypoxia, or the impairment of myocardium in patients with coronary artery disease (CAD). To assess potential role of plasma lipoproteins in O₂-demanding tissues, the Bruce protocol was employed as a standardized treadmill test, as well known in the art.

6 healthy volunteers (HV, 42-61 years old) and 4 CAD patients, eligible for the treadmill test (age 45-59 years old) were recruited for this study. Apart from assessing changes their electrocardiogram (ECG), pulse and blood pressure, samples and level of oxygen carrying capacity of plasma lipoproteins, OCCL, from venous blood was analyzed prior to the test and within 3-4 minutes afterward. The measurement was conducted using a catalymetry assay. In short, 10 μl of citrate plasma was added to 0.98 ml cuvette of 0.05 M phosphate buffer with 0.1 mM sodium ethylenediamine tetraacetic acid (EDTA-Na), containing 10⁻⁶ M phenazine methosulphate and 10⁻⁴ M nitro blue tetrazolium chloride (NBT). The reaction was initiated by adding 10 μl of 10⁻⁴ M NADH.

The difference in absorption at λ=560 nm between starting point and after 60 seconds was measured by spectroscopy. This difference was used to measure oxygen-depended reduction of NBT. The results of this reaction were compared with the control reaction where instead of plasma 10 μl of the reaction buffer was added. The difference in the reduction of NBT with the analyzed sample and the control was used to calculate the amount of O₂ in added plasma lipoproteins.

The results of the described study are presented in the Table 5, below. It was found that in healthy persons, after the test, the OCCL level significantly dropped in the circulating blood indicating that oxygen carrying lipoproteins were taken up by O₂-demanding exercising tissues. In patients with CAD, on the other hand, this change in the plasma oxygen level was not observed, indicating that the CAD-patients did not (or could not) take up lipoprotein oxygen from circulation.

TABLE 5
Changes in OCCL in venous during the treadmill
test in patients with CAD and healthy volunteers

| | OCCL, in ΔE at λ = 560 nm × 10³ | |
|---|---|---|
| Clinical groups | Before test | After test |
| Healthy volunteers | 262 ± 15.1 | 85 ± 3.4 |
| | | p < 0.01 |
| CAD patients | 330 ± 19.3 | 345 ± 20.2 |
| | p (CAD − HV) > 0.05 | p > 0.05 |
| | | p (CAD − HV) < 0.05 |

### Example 2.2—Clinical Hypoxic Pathologies

To assess the physiological role of lipoprotein oxygen, the O₂ level in the blood of patients was measured at different time points after (and sometimes prior) to various types of acute hypoxic events.

The patient group comprised of 10 patients with unstable angina (age 52-70 years old), 9 patients with acute myocardial infarction, (age 49-65 years old), and 4 patients with ischemic stroke (IS, age 56-75 years old). Additionally, 2 patients (males 5 and 56 years old) participated, who at the time of admission to the clinic had stable CAD, but developed acute myocardial infarction on the following day.

The results of OCCL changes in the blood of all these patients are presented in Table 6 below.

TABLE 6
Changes in OCCL in different hypoxic events

| | OCCL in ΔE at λ = 560 nm × 10³ | | | |
|---|---|---|---|---|
| Patient | 1 day before | Days after acute hypoxic event | | |
| groups | hypoxic event | 1 | 8 ± 1* | 20 ± 1** |
| CAD | 150 | 82 | 177 | 417 |
| patient 1 | | | | |
| patient 2 | 380 | 260 | 400 | 560 |
| Unstable | | 200 | 287 | 378 |
| angina | | | p (1-8) > 0.05 | p (1-20) < 0.005 |
| | | | | p (1-8) < 0.01 |
| Acute | | 118 | 254 | 397 |
| myocardial | | | p(1-8) < 0.01 | p (1-20) < 0.001 |
| infarction | | | | p (1-8) < 0.005 |
| Ischaemic | | 306 | 332 | 369 |
| stroke | | | p (1-8) > 0.05 | p (1-20) > 0.05 |
| | | | | p (1-8) > 0.05 |

*average of 7-10 days,
**average of 20-22 days

These data demonstrate that across all patients, the level of lipoprotein oxygen was the lowest at the time closest to the acute hypoxia event. After this point, the level of lipoprotein oxygen increased gradually. This dynamic was less prominent in the case of the ischemic stroke than in patients with cardiovascular acute events, probably due to the fact the hypoxia in the brain does not affect blood supply as badly as hypoxia in the heart.

Acute tissue hypoxia, such as myocardial infarction are characterized not only by restricted oxygen delivery to the part of the heart tissue, supplied by the clotted coronary artery, but by an impairment of the activity of the whole heart. Consequently, negatively reduced blood flow in the body occurs, and functions of many organs are compromised. As a result, this local hypoxic crisis leads to the systemic body hypoxia.

### Example 2.3—Uniqueness of OCCL as the Only Blood Test Measuring Tissue Oxygenation and its Hypoxia

### Example 2.4—Lipid Oxygen Treatment

In this section the effects of regular intake of validated bioactive nutraceuticals on blood OCCL and tissue oxygen saturation (StO₂) was evaluated.

Since oxygen solubility is higher in lipids than in aqueous media, it was interesting to examine a possible link between changes in OCCL and lipoprotein concentration in blood. For these purposes one group of participants was given LDL (low-density lipoprotein)-lowering drug, simvastatin.

For this study 72 clinically healthy volunteers were recruited (age 35-57 years old), 30 male and 34 female). The subjects had not taken any medication or dietary supplements prior to the experiment. The volunteers were randomized and divided into 7 groups of 8 persons each. The participants were given a one-month supply of a particular nutraceutical or pharmaceutical. The participants did not know which nutraceutical or pharmaceutical was given to them. The daily dose for each product was taken in 1 capsule with the main meal of the day. The blood and StO₂ were analyzed prior the trial and after (4 weeks). OCCL was analyzed as described above.

Thenar eminence and forearm muscles of the patients were used as a tissue target for the assessment of StO₂, or combined level of oxygenated hemoglobin and myoglobin. StO₂ was assessed by continuous wavelength near-infrared spectroscopy, NIRS, with wide-gap second-derivative (In Spectra, Hutchinson Technology, MN, USA), as known in the art. The measurements were taken at different time points. The recording was initiated after 15 min of rest in a supine position, before occlusion of the brachial artery. It was then continued during stagnant ischemia induced by rapidly inflating a cuff to 50 mm Hg above systolic BP. The ischemia lasted for 3 min, and the recording period lasted for an additional 5 min, until StO₂ was stabilized

The area under the hyperemic curve, AUC, of the recorded signal for the settling time in the post occlusion period was then calculated in AUC mm, or % O₂/min. All body and vascular parameters were recorded in the morning between 8 and 10 a.m.

The results of this study are summarized in Table 7. Out of 7 tested product regular intake of 4 products, 3 different carotenoids and Q10, resulted in an increase in OCCL, which in turn translated into significant improvement of peripheral tissue oxygenation.

Supplementation with two other nutraceuticals, DHA Omega 3 and vitamin D3, and treatment with simvastatin did not affect neither OCCL nor StO₂ level.

Importantly, the changes in the lipoprotein concentration were not related to changes in OCCL level, indicating that the quantity of lipids are not important for their ability to carry oxygen, but rather their ability to carry/capture oxygen.

TABLE 7
Increase in plasma lipoprotein oxygen translated into improvement of tissue oxygen saturation.

| | | | Oxygen Parameters | | | |
|---|---|---|---|---|---|---|
| | | | OCCL in | | StO₂ in | |
| Products | LDL in mg/dL | | ΔE at λ = 560 nm × 10³ | | AUC mm | |
| daily dose | 0 w | 4 w | 0 w | 4 w | 0 w | 4 w |
| DHA | 155 ± 9.2 | 154 ± 7.5 | 626 ± 34 | 639 ± 42 | 74 ± 0.71 | 76 ± 0.92 |
| Omega 3 250 | | p > 0.05 | | p > 0.05 | | p > 0.05 |
| mg | | | | | | |
| Lycopene | 156 ± 6.1 | 135 ± 7.0 | 648 ± 41 | 786 ± 39 | 75 ± 0.73 | 85 ± 0.61 |
| 7 mg | | p < 0.05* | | p < 0.05* | | p < 0.05* |
| Lutein & | 160 ± 10.2 | 159 ± 11.4 | 432 ± 50 | 544 ± 51 | 75 ± 0.82 | 83 ± 0.91 |
| Zeaxanthin | | p > 0.05 | | p < 0.05* | | p < 0.05* |
| 10 mg:2 mg | | | | | | |
| Astaxanthin | 153 ± 7.3 | 151 ± 6.2 | 539 ± 52 | 770 ± 57 | 77 ± 0.74 | 87 ± 0.84 |
| 7 mg | | p > 0.05 | | p < 0.05* | | p < 0.05* |
| Coenzyme | 143 ± 5.7 | 140 ± 4.2 | 505 ± 66 | 593 ± 64 | 75.0 ± 7.4 | 84.0 ± 8.5 |
| Q10 100 mg | | p > 0.05 | | p < 0.05* | | p < 0.05* |
| Vitamin D3 | 156 ± 8.4 | 144 ± 5.9 | 412 ± 48 | 443 ± 51 | 66.8 ± 6.0 | 71.2 ± 6.8 |
| 4,000 IU | | p > 0.05 | | p > 0.05 | | p > 0.05 |
| Simvastatin | 163 ± 8.2 | 129 ± 7.4 | 451 ± 51 | 504 ± 53 | 70 ± 0.72 | 73 ± 0.81 |
| 40 mg | | p < 0.01* | | p > 0.05 | | p > 0.05 |

*statistically significant difference

### Example 2.5—Lipid Oxygen Treatment to Induce Cardiovascular Improvement

In the following study, the increase of OCCL in the blood and tissue oxygen saturation (StO₂) in peripheral tissue and its possibility in affecting the physiological body parameters in patients with ischemic pathologies was assessed.

120 patients with stable CAD were recruited for the study, 68 male and 52 female (42-76 years old). The subjects were randomized on life-style, body base-line parameters and prescribed medication, and divided into two equal groups. Patients in one group received 7 mg of highly bioavailable lycopene (GA Lycopene) in 1 capsule per day. Patients in the other group received 7 mg of control lycopene with poor bioavailability. The participants were instructed to take 1 capsule per day with their main meal. The trial was double blind, in that neither patients nor investigators knew which products were given to which group.

Blood and physiological parameters were analyzed before the study and after 4 weeks. All body and vascular parameters were recorded in the morning between 8 and 10 a.m.

Pulse rate, systolic and diastolic blood pressure, SBP and DBP, were recorded three times on the left arm of the seated patient after 15 min of rest. The time between measurements was greater than 2 min. The mean result for each parameter was calculated and endothelium-dependent flow-mediated (FMD) vasodilatation was measured in accordance with widely accepted guidelines. Patients were screened under ambient conditions at the same time of the morning in a supine position.

High-resolution ultrasound was applied at the same anatomical landmark of a section of the brachial artery for a period of 30 s before and during the peak of reactive hyperemia. It was positioned prior to sphygmomanometer cuff occlusion and 1 min after its deflation. The level of inflation was 50 mm Hg above the patient's systolic blood pressure, and continued for 5 min. Arterial diameter was imaged above the antecubital fossa in a longitudinal scan by duplex ultrasound with linear phase-array transducer. FMD was calculated as a change in post-stimulus diameter as a percentage of the baseline diameter, as described in the art.

Ankle-Brachial Index, ABI, was measured between left and right brachial arteries, the one with the highest SBP was chosen, and between left and right tibial arteries, the one with the highest SBP was also chosen. For this purpose, a continuous-wave Doppler probe was used after patients had been in a supine position for at least 15 min of rest, as described in the art. The results of this study are presented in Table 8. As seen from the table, in the group that received highly bioavailable lycopene, the OCCL level in blood plasma was significantly increased, while the OCCL level in the group that received the same amount of control lycopene, remained unchanged.

These changes in the blood in the former group were accompanied by increase in their tissue oxygenation, which was not observed in the latter group.

TABLE 8
Increase in plasma lipoprotein oxygen and tissue respiration translated
into improvement of improvement of cardiovascular parameters

| Analysed | Control lycopene | | GA Lycopene | |
|---|---|---|---|---|
| parameters | 0 w | 4 w | 0 w | 4 w |
| OCCL in μM O₂ | 3.86 ± 0.32 | 3.77 ± 0.27 | 3.67 ± 0.29 | 5.27 ± 0.59 |
| | | p > 0.05 | | p < 0.01** |
| StO₂ (% O₂/min) | 12.6 ± 1.1 | 12.9 ± 1.2 | 11.9± 1.0 | 13.8 ± 1.1 |
| | | p > 0.05 | | p < 0.05** |
| Pulse rate | 70 ± 0.81 | 69 ± 0.75 | 75 ± 0.69 | 74 ± 0.65 |
| | | p > 0.05 | | p > 0.05 |
| Systolic BP | 120 ± 3.6 | 125 ± 4.1 | 122 ± 2.7 | 119 ± 2.0 |
| | | p > 0.05 | | p < 0.05** |
| Diastolic BP | 73 ± 2.9 | 73 ± 3.5 | 75 ± 5.9 | 79 ± 6.6 |
| | | p > 0.05 | | p > 0.05 |
| ABI | 1.1 ± 0.08 | 1.1 ± 0.09 | 0.95 ± 0.05 | 0.93 ± 0.04 |
| | | p > 0.05 | | p < 0.05** |
| FMD | 11.2 ± 1.3 | 11.1 ± 1.0 | 10.3 ± 0.2 | 11.4 ± 0.3 |
| | | p > 0.05 | | p < 0.05** |

*in ΔΕ at λ = 560 nm × 10³
**statistically significant

Importantly, the increase in OCCL and StO₂ translated into improvement of a number of cardiovascular parameters in these patients, including reduction of the systolic blood pressure and ABI, and increase in their FMD.

The results of these clinical trials thus indicate that the increase in OCCL can lead to increase in oxygen supply to peripheral tissues, which in turn improves tissue oxygenation.

Importantly, the increase in lipid oxygenation leads to boost in StO₂ and tissue respiration, which in turn advantageously improves cardiovascular parameters in patients with one of the world's leading hypoxic pathology, CAD.

These observations provide strong rationale to use lipid oxygen as a new treatment target to support body tissue oxygenation in health and work, to provide its resistance to and ability to cope with stress, to prevent and to treat hypoxic conditions and pathologies.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-57. (canceled)

58. A device for measuring a concentration of molecular oxygen in a biological sample, the device comprising a housing comprising a plurality of membranes, wherein the plurality of membranes comprises a separation membrane configured to separate components in the biological sample, and a reagent membrane configured to facilitate an oxygen dependent reaction, wherein the oxygen dependent reaction is indicative of the concentration of molecular oxygen in the biological samples.

59. The device of claim 58, wherein the separation membrane comprises a first membrane and a second membrane, wherein the first membrane comprises a D23 membrane and wherein the second membrane comprises a mixed matrix membrane (MMM).

60. The device of claim 58, wherein the plurality of membranes further comprises a blood spreading membrane.

61. The device of claims 58, wherein the reagent membrane comprises an oxidating agent and a reducing agent, wherein the oxidizing agent comprises a quinone derivative and wherein the quinone derivative comprises menadione.

62. The device of claim 58, wherein the device is configured such that the biological samples flow vertically from the blood spreading membrane, through the first membrane and second membrane to the reagent membrane.

63. The device of claim 58, configured to indicate an oxygen carrying capacity, oxygen carrying capacity reserve or oxygen take up ability in the biological sample.

64. The device of claim 58, for use in assessment of predisposition or resistance to hypoxic conditions or diseases or for use in diagnosis of hypoxia-associated asymptomatic or symptomatic pathologies.

65. The device of claim 58, for use in assessment and monitoring of effects of an administered dietary supplement, nutraceuticals, pharmaceutical, or medical procedure, dietary or life-style factor, or a combination thereof.

66. The device of claim 58, for use in development of a functional food or beverage, nutraceutical, or pharmaceutical product configured to support tissue oxygenation, to prevent or to treat tissue hypoxic conditions.

67. The device of claim 58, for use in assessment of integrity or quality of a lipid containing food, beverage, nutraceutical, pharmaceutical, or industrial products.

68. A method for measuring or monitoring a lipid or lipoprotein oxygen concentration and an oxygen uptake capability of a subject, the method comprising measuring an oxygen carrying capacity of plasma lipoproteins (OCCL) in a biological sample of the subject, using a biochemical, electro-chemical, chemical or physical method or assay.

69. The method according to claim 68, further comprising a blood test measuring a level of tissue oxygenation, tissue oxygen supply configured to detect oxygen deficiency, impairment or hypoxia on a symptomatic or asymptomatic level.

70. The method of claim 68, wherein the OCCL level is measured before and after changing an oxygen-related condition in the subject, wherein a change or lack thereof in the level of OCCL in response to the change in the oxygen-demanding condition is indicative of the subject's tissue oxygen delivery or tissue oxygenation level.

71. The method of claim 68, further comprising subjecting the biological sample to a reagent configured to facilitate an oxygen dependent reaction, the oxygen dependent reaction indicative of the concentration of molecular oxygen in the biological samples.

72. The method of claim 68, wherein changing the oxygen-related condition comprises increasing a tissue oxygen demanding physical work, mental work, physical stress or mental stress on the subject, wherein increasing the stress on the subject comprises physical work or exercise or inducing a transient stagnant ischemia test.

73. The method of claim 68, wherein the subject is pregnant or wherein the subject suffers from coronary artery disease, unstable angina, acute myocardial infarction, peripheral tissue or organ ischemia.

74. A method for preventing, ameliorating or treating a condition characterized by insufficient tissue oxygenation, the method comprising administering to a subject in need thereof a therapeutic agent capable of increasing OCCL in a subject.

75. The method of claim 74, wherein the subject shows less than a 10% increase in the OCCL when subjected to a change in an oxygen-related condition, wherein measuring the OCCL comprises performing a biochemical, electro-chemical, chemical or physical method or assay on a biological sample obtained before and after the subjecting of the subject to the change in the oxygen tissue supply or tissue oxygenation conditions, wherein the measuring comprises subjecting the biological samples to a reagent configured to facilitate an oxygen dependent reaction, the oxygen dependent reaction indicative of the concentration of molecular oxygen therein.

76. The method of claim 74, where the therapeutic agent is administered orally.

77. The method of claim 74, where the therapeutic agent is Lycopene, Lutein, Zeaxanthin, Astaxanthin, Coenzyme Q10 or any combination thereof.
